(19) United States
(12) Patent Application Publication
(10) Pub. No.: US 2009/0258071 A1
(43) Pub. Date: Oct. 15, 2009

(54) COMPOSITIONS AND METHODS FOR PH TARGETED DRUG DELIVERY

(75) Inventors: David Lessard, Montreal (CA); Laibin Luo, Pierrefonds (CA); Dorothee Le Garrec, Montreal (CA); Damon Smith, Saint-Laurent (CA)

Correspondence Address: GOODWIN PROCTER LLP;PATENT ADMINISTRATOR, 53 STATE STREET, EXCHANGE PLACE, BOSTON, MA, 02109-2881, US

(73) Assignee: Labopharm, Inc., Laval (CA)

(21) Appl. No.: 12/408,481

(22) Filed: Mar. 20, 2009

Related U.S. Application Data
(63) Continuation-in-part of application No. PCT/IB2007/004171, filed on Sep. 24, 2007.
(60) Provisional application No. 60/846,355, filed on Sep. 22, 2006.

(51) Int. Cl. A61K 9/14; A61K 47/34; A61K 31/437; A61P 35/00

(52) U.S. Cl. 424/489; 514/772.1; 514/283; 977/906

(57) ABSTRACT

The invention provides compositions and methods for the targeted, in particular, pH targeted, delivery of pharmaceutically active agents in mammals. The compositions comprise pH sensitive diblock copolymers, which permit the release of the pharmaceutically active agent when exposed to an environment having a particular pH. The compositions are particularly useful for the oral delivery of water insoluble pharmaceutically active agents.

### RELATED APPLICATIONS

This application is a continuation-in-part of International Patent Application Serial No. PCT/IB07/004,171, which claims the benefit of and priority to U.S. Patent Application No. 60/846,355, filed Sep. 22, 2006, the disclosures of each of which are incorporated by reference herein.

### FIELD OF THE INVENTION

The invention relates generally to compositions and methods for the targeted delivery of pharmaceutically active agents, and more particularly, the invention relates to compositions and methods for pH targeted delivery of pharmaceutically active agents.

### BACKGROUND

A number of approaches have been developed for the delivery of pharmaceutically active agents in a mammal. The objective is to deliver the pharmaceutically active agents to a site in the mammal where they can impart their pharmacological effect. It is appreciated, however, that for certain agents, there are benefits in site specific delivery which may be mediated by environmental pH. For example, this can be helpful for oral administration where the active ingredient needs to be protected from the acidic environment of the stomach but then made available for absorption once the agent passes out of the stomach and into the large intestines. One approach, for example, includes coating capsules or tablets with a pH sensitive polymer, for example, Eudragit®, which maintains the integrity of the capsules or tablets while passing through the stomach but dissolves as the pH increases in the intestines. These coatings, however, do not improve the solubility of water insoluble drugs contained within the capsules or tablets.

As a result, there is still a need for other pH targeted drug delivery systems.

### SUMMARY OF THE INVENTION

The invention is based, in part, upon the discovery that it is possible to produce compositions comprising pH sensitive diblock copolymers that increase the solubility of water insoluble pharmaceutically active agents and deliver the active agents in a pH dependent manner so as to increase their bioavailability in mammals. The compositions, when exposed to a pH permissive environment, for example, at a pH greater than about 4, release the pharmaceutically active agent for absorption within the mammal. The compositions are particularly useful for oral drug delivery. When present in the stomach, the compositions do not release a substantial amount (for example, less than 10%) of the pharmaceutically active agent. However, as the compositions leave the stomach and enter the large intestines, the compositions, as a result of the increase in phi, start to release the pharmaceutically active agent in a pH dependent manner.

In one aspect, the invention provides a composition for the pH targeted delivery of a water insoluble pharmaceutically active agent. The composition comprises (a) a plurality of pH sensitive diblock copolymers; and (b) a water insoluble pharmaceutically active agent associated with the diblock copolymers. The composition is further characterized in that, when in contact with an aqueous solution at a pH of about 2, less than about 10% of the pharmaceutically active agent is released from the composition after 2 hours, but when in an aqueous solution of the same or similar composition having a pH of at least 6 or higher, at least 60% of the pharmaceutically active agent is released from the composition within 2 hours. It is understood that the composition can be administered in a dry form, for example, in a tablet, or in a physiologically acceptable solution or suspension.

In another aspect, the invention provides a pH-sensitive micellar composition for the targeted delivery of a water insoluble pharmaceutically active agent. The composition comprises: (a) micelles comprising a plurality of pH sensitive dibock copolymers; and (b) a water insoluble pharmaceutically active agent disposed within the micelles. When contacted with an aqueous solution at a pH of about 2, less than about 10% of the pharmaceutically active agent is released from the micelles after 2 hours. However, when present in the same or a similar aqueous solution at a pH of at least 6 or higher, at least 60% of the pharmaceutically active agent is released from the micelles within 2 hours. Under certain circumstances, at least 70%, or at least 80%, of the pharmaceutically active agent is released from the micelles within 2 hours.

The diblock co-polymers comprise a first block and a second block. In one embodiment, the first block of the diblock copolymer comprises monomers selected from the group consisting of poly(ethyleneglycol) and poly(vinylpyrrolidone). The second block of the diblock co-polymer comprises a combination of (i) ionizable monomers selected from the group consisting of methacrylic acid and acrylic acid, and (ii) hydrophobic monomers selected from the group consisting of methacrylate and derivatives thereof, acrylates and derivatives thereof, methacrylamides, and acrylamides.

In one embodiment, the preferred polymer is a block co-polymer, wherein the first block comprises ethyleneglycol monomer subunits and the second block comprises monomer subunits of both methacrylic acid and n-butylmethacrylate. In the second block, the monomer subunits generally are randomly organized. For example, the monomer subunits can be arranged such that the methacrylic acid monomer subunits or strings of methacrylic acid monomer subunits are interspersed between the n-butylmethacrylate monomer subunits or strings of n-butylmethacrylate monomer subunits or vice versa. Exemplary diblock copolymers are defined by Formula I.

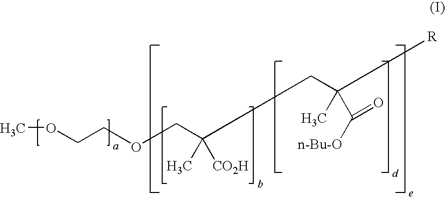

wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0.

In another aspect, the invention provides a composition comprising:

(a) a plurality of pH sensitive diblock copolymers, wherein the diblock copolymers are defined by Formula I,

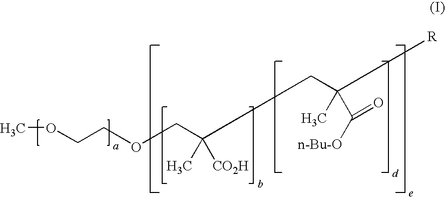

wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0; and

(b) a camptothecin derivative, for example, SN-38, associated with the diblock copolymers. In certain embodiments, the composition includes a therapeutically effective amount of the camptothecin derivative.

In another aspect, the invention provides a method of producing pH sensitive compositions for pH targeted drug delivery. In one approach, the method comprises (a) producing a solution comprising pH sensitive diblock copolymers, for example, the copolymers discussed above, and a water insoluble pharmaceutically active agent; and (b) drying the solution of step (a) to produce a dried product.

In one embodiment, the solution produced in step (a) has a pH greater than about 7. Under certain circumstances, it can be advantageous to adjust the pH to a pH in the range from about 5 to about 7 prior to drying the solution to produce a dried product. In addition, in one approach the pharmaceutically active agent and the diblock copolymers are solubilized in different solvents before they are combined to produce the solution of step (a). In another approach, the pharmaceutically active agent and the diblock copolymers are solubilized in separate and distinct portions of the same solvent before they are combined to produce the solution of step (a).

In another aspect, the invention provides a method of administering an effective amount of a water insoluble pharmaceutically active agent to a mammal, for example, a human, in need thereof. The method comprises administering one or more of the compositions described herein so as to administer an effective amount of the pharmaceutically active agent. It is understood that the compositions can be administered orally or parenterally. It is appreciated, however, that the compositions are particularly useful in oral administration wherein the water insoluble pharmaceutically active agent is protected from stomach acid but then is preferentially delivered and absorbed once the composition has passed out of the stomach and into the intestines where the pH is higher than in the stomach. It is also appreciated that the composition can be administered in a dry form, as a suspension, or in a solution.

These and other aspects and features of the invention are described in the following figures, detailed description and claims.

### BRIEF DESCRIPTION OF THE DRAWINGS

The invention is illustrated but is not limited by the annexed drawings, in which

FIG. 1 is a schematic representation of an exemplary pH sensitive micellar composition;

FIG. 2 is a schematic representation showing how the compositions of the invention transition as a function of pH;

FIG. 3 is a graph showing the dissolution profile of a micellar composition of the invention containing the camptothecin derivative SN-38 in an aqueous medium at pH 1.2;

FIG. 4 is a graph showing the dissolution profile of SN-38 either alone (—▪—) or from a micellar composition of the invention (——) in an aqueous medium at pH 6.8;

FIG. 5 is a graph showing the pharmacokinetics in CD1 mice of SN-38 administered either alone (——) or as an SN-38 containing micellar composition (—∘—);

FIG. 6 is a graph showing the maximum tolerated dose of SN-38 in mice following administration of phosphate buffer (——), 25 mg/kg of SN-38 containing micelles (—▪—), and 50 mg/kg of SN-38 containing micelles (—▴—);

FIG. 7 is a graph showing the efficacy of micellar compositions containing SN-38 on reducing tumor volume in Swiss nude mice administered with phosphate buffer (——), 25 mg/kg of SN-38 containing micelles (—▪—), 50 mg/kg of SN-38 containing micelles (—▴—), and 100 mg/kg of SN-38 containing micelles (—♦—);

FIG. 8 is a graph showing the efficacy of micellar compositions containing SN-38 (SN38-PNDS) on HCT-116 colorectal carcinoma tumor volume in Swiss nude mice following administration of a vehicle (phosphate buffer) (——), 50 mg/kg CPT-11 (—□—), 75 mg/kg SN38-PNDS (—∘—), and 25 mg/kg SN38-PNDS (—Δ—);

FIG. 9 is a graph showing the efficacy of micellar compositions containing SN-38 on HCT-116 colorectal carcinoma tumor volume in Swiss nude mice following administration of a vehicle (——), 12.5 mg/kg SN38-PNDS (—*—), 25 mg/kg SN38-PNDS (—∘—), 50 mg/kg SN38-PNDS (—□—), 75 mg/kg SN38-PNDS (—Δ—), and 50 mg/kg CPT-11 (—▪—);

FIG. 10 is a bar chart showing the permeability of micellar compositions containing SN-38 (SN38-PNDS) across Caco-2 monolayers as compared to SN-38 solubilized in DMSO; and

FIG. 11 is a bar chart showing the levels of SN-38 and SN-38 glucoronide metabolite upon administration of CPT-11 intravenously and micellar compositions containing SN-38 (SN38-PNDS) orally to Sprague-Dawley rats.

### DETAILED DESCRIPTION

The invention is based, in part, upon the discovery that it is possible to produce a targeted delivery system using pH sensitive micelles to deliver water insoluble pharmaceutically active agents to a mammal, for example, a human. The compositions are particularly useful for the delivery of water insoluble pharmaceutically active agents, for example, the camptothecin derivative, SN-38.

The pH targeted delivery system is stable at low pH, for example, in the range of about 1 to about 4 and does not release a significant amount, for example, less than 10% of the pharmaceutically active agent within this pH range for a prolonged period of time, for example, after one or two hours. The pH of the stomach of a mammal can be in the range of about 1 to about 4. Accordingly, it is contemplated that the compositions of the invention are stable in the stomach and, therefore, do not release a significant amount of the pharmaceutically active agent as the compositions pass through the stomach. Once the compositions leave the stomach and enter the upper and lower intestines, the pH of the surrounding environment increases. In the range of from about pH 4 to about pH 6, the compositions of the invention start to release the pharmaceutically active agent disposed therein. As a result, the drug is released from the compositions to permit absorption within the intestines.

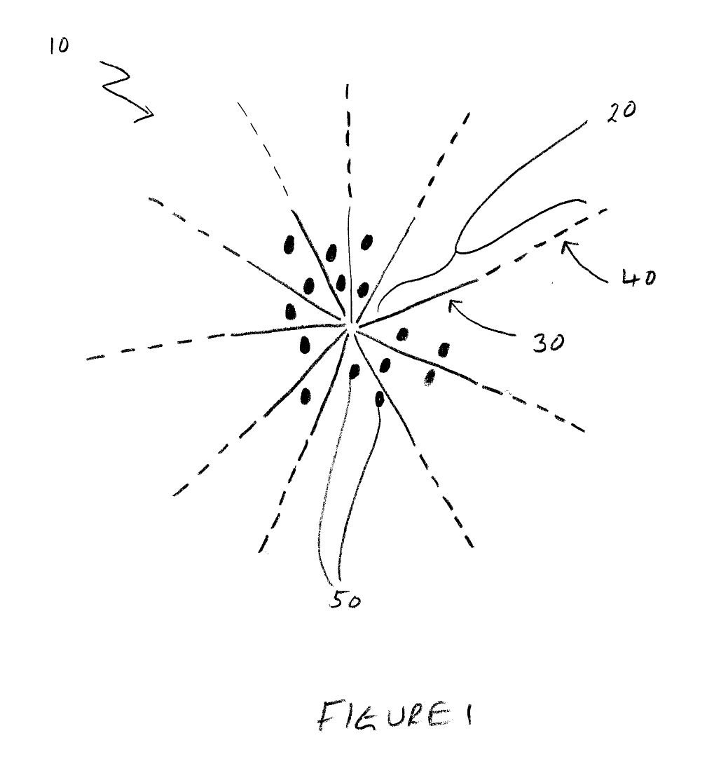

Exemplary micellar compositions are shown schematically in FIG. 1. In particular, an exemplary micelle 10 comprises a plurality of pH sensitive polymers 20 each of which contain a hydrophobic portion 30 and a hydrophilic portion 40. In certain embodiments, the hydrophilic portion 40 is defined by a pH sensitive (for example, an anionizable) polymer. The hydrophobic portions 30 together define a hydrophobic core of micelle 10. The hydrophilic portions 40 together define a hydrophilic exterior of the micelle 10. Water insoluble pharmaceutically active agent 50 is shown to be distributed preferentially within the hydrophobic core of micelle 10.

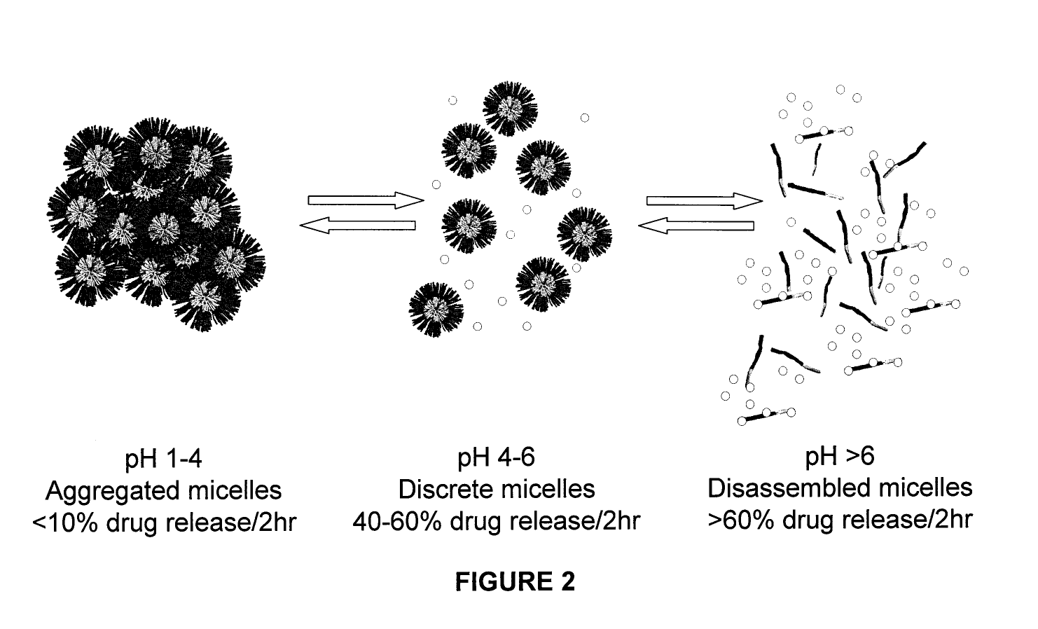

The performance of the compositions of the invention as a function of the pH is shown schematically in FIG. 2. In the range of pH1 to pH4, the micelles generally are aggregated in solution and, under these conditions, the aggregated micelles typically release less than 10% by weight of the drug disposed within the micelles in 2 hours. In the range of pH 4 to pH 6, the aggregated micelles disaggregate to produce discrete micelles, and under these conditions the discrete micelles release from about 40% by weight to about 60% by weight of the drug disposed within the micelles in 2 hours. At a pH greater than 6, the discrete micelles disassemble releasing the diblock copolymers and the pharmaceutically active agent, and under these conditions the disassembled micelles release greater than 60% by weight of drug within 2 hours. As shown in FIG. 2, each of the three morphological states are reversibly interchangeable with one another as a function of pH. As a result of these properties, the pH targeted delivery system is a stable aggregate at low pH, for example at a pH between 1 and 2 (as found in the stomach) and does not release a significant amount, for example, less than 10% of the pharmaceutically active agent after 2 hours. Once the compositions leave the stomach and enter the upper intestine, the pH of the surrounding environment increases. In the range of pH 4 to 6, the aggregated micelles start to disaggregate into single micelles, which may adhere to the mucous membrane of the wall of the gastrointestinal tract. It is believed that significant drug release occurs at this point. As the pH increases further, as can happen in the intestines, the micelles disassemble to release the remainder of the drug in the molecular form most suitable for absorption across the wall of the intestines.

The choice of suitable pharmaceutically active agents, diblock copolymers, methods of making the compositions of the invention, and dosing and administration of the compositions of the invention are discussed in the following sections.

### I. Pharmaceutically Active Agents

It is understood that the compositions of the invention can be used to deliver one or more water insoluble pharmaceutically active agents.

The term “pharmaceutically active agent” refers to any chemical moiety that is a biologically, physiologically, or pharmacologically active substance that acts locally or systemically in a subject. Also embraced are salts and prodrugs of the pharmaceutically active agent. Examples of pharmaceutically active agents, also referred to herein as “drugs,” are described in well-known literature references such as the Merck Index, the Physicians Desk Reference, and The Pharmacological Basis of Therapeutics, and include, without limitation, medicaments; vitamins; mineral supplements; substances used for the treatment, prevention, diagnosis, cure or mitigation of a disease or illness; substances which affect the structure or function of the body. As used herein, the term “water insoluble pharmaceutically active agent” is understood to mean a pharmaceutically active agent that has a solubility of 1 mg/mL or less in water.

Compositions and formulations contemplated herein may include one or more pharmaceutically active agents. For example, a composition may include two, three or more different pharmaceutically active agents.

The pharmaceutically active agents can vary widely with the purpose for the composition. Non-limiting examples of broad categories of useful pharmaceutically active agents include the following therapeutic categories: anabolic agents, anti-cancer agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, cerebral dilators, coronary dilators, decongestants, diuretics, diagnostic agents, hyperglycemic agents, hypnotics, hypoglycemic agents, neuromuscular drugs, peripheral vasodilators, psychotropics, sedatives, stimulants, thyroid and anti-thyroid agents, uterine relaxants, vitamins, and pro-drugs.

In certain embodiments, the pharmaceutically active agent is an anti-cancer agent. Exemplary anti-cancer agents that can be incorporated into the delivery systems described herein include, for example, amsacrine, anagreline, anastrozole, bicalutamide, bleomycin, busulfan, camptothecin, camptothecin derivatives, carboplatin, carmustine, chlorambucil, cisplatin, dactinomycin, dexamethasone, estramustine, etoposide, fludrocortisone, megestrol, melphalan, mitomycin, temsirolimus, teniposide, taxanes, testosterone, tretinoin, vinblastine, vincristine, vindesine and vinorelbine. Exemplary camptothecin derivatives include, for example, 10-hydroxy-camptothecin, 7-ethyl-10-hydroxy-camptothecin (also known as SN-38), topotecan, 9-aminocamptothecin, 9-nitrocamptothecin, 10,11-methylenedioxycamptothecin, 9-amino-10,11 methylenedioxycamptothecin, 9-chloro-10,11-methylene-dioxycamptothecin. Exemplary taxanes include, for example, palitaxel and docetaxel.

### II. Diblock Copolymers

The drug delivery systems described herein are pH sensitive and, as discussed herein, release the pharmaceutically active agents in a pH dependent manner. The pH sensitivity is based, in part, upon the particular diblock copolymers used in the compositions.

The diblock co-polymers comprise a first block and a second block. In one embodiment, the first block of the diblock copolymer comprises monomers selected from the group consisting of poly(ethyleneglycol) and poly(vinylpyrrolidone). The second block of the diblock co-polymer comprises a combination of (i) ionizable monomers selected from the group consisting of methacrylic acid and acrylic acid, and (ii) hydrophobic monomers selected from the group consisting of methacrylate and derivatives thereof, acrylates and derivatives thereof, methacrylamides, and acrylamides. Exemplary polymers and polymer subunits are described in U.S. Pat. No. 6,939,564.

In one embodiment, the preferred polymer is a block co-polymer, wherein the first block comprises ethyleneglycol monomer subunits and the second block comprises monomer subunits of both methacrylic acid and n-butylmethacrylate. In the second block, the monomer subunits generally are randomly organized. Exemplary diblock copolymers are defined by Formula I

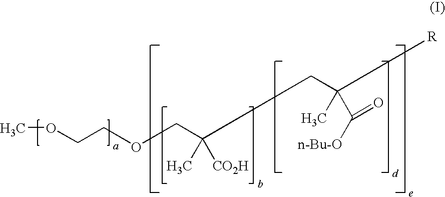

wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 75 but more preferably in the range from about 10 to about 50, and
provided that at least one occurrence of b is >0, and at least one occurrence of d is >0.

In addition, exemplary diblock copolymers are defined by Formula II, wherein the first block comprises ethyleneglycol monomeric subunits and the second block comprises randomly arranged monomeric subunits of methacrylic acid (denoted as B) and n-butylmethacrylate (denoted as C). It is understood that the monomeric subunits of methacrylic acid (B) and n-butylmethacrylate (C) in the second block can be randomly positioned in the form of, for example, BBCC, BCBC, BCCB, CBCB, CBBC, and CCBB.

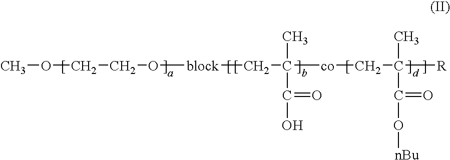

wherein,
R is H, alkyl, hydroxyl, alkoxyl, or halogen,
a is an integer in the range of about 20 to about 60,
b represents independently for each occurrence an integer in the range of 30 to about 120, and
d represents independently for each occurrence an integer in the range of 10 to about 50.

In another embodiment, a preferred diblock copolymer has a first block comprising 20-60 (preferably 40-50, more preferably 45) ethyleneglycol monomer subunits covalently linked to a second block comprising a random arrangement of 30-120 (preferably 40-110) methacrylic acid monomer subunits and 10-50 (preferably 20-40) n-butylmethacrylate monomer subunits. This polymer is referred to herein as [poly(ethyleneglycol)]-poly[(methacrylic acid)-(n-butyl methacrylate)] or PEG-PMA. Exemplary polymers useful in the practice of the invention are described in more detail in Example 1.

The foregoing polymers can be created using the synthetic protocols set forth in SCHEMES 1 and 2.

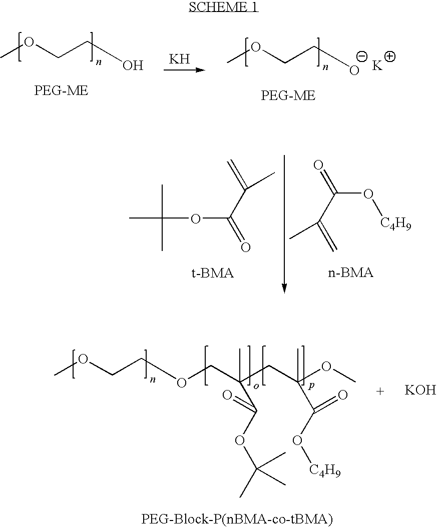

Briefly, poly(ethyleneglycol) (PEG) (MW 2,000) is dissolved in tetrahydrofuran (THF) and reacted with potassium hydride (KH). Then, tert-butyl methacrylate (t-BMA) and n-butyl methacrylate (n-BMA) are added to the reaction mixture, which is then reacted for 2 hours at 20° C. The resulting PEG-block-P(nBMA-co-tBMA) copolymer is collected following solvent evaporation and then is subjected to hydrolysis according to SCHEME 2.

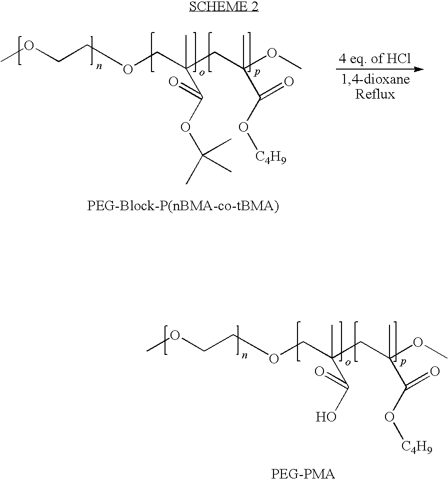

For example, the PEG-block-P(nBMA-co-tBMA) from SCHEME 1 is combined with 1,4-dioxane and hydrochloric acid (1-HCl), and refluxed overnight. After cooling, the solvent is removed and the product dissolved in THF. The product then is precipitated in cold water and harvested by centrifugation. The product then is twice resuspended in THF, precipitated and harvested by centrifugation. The resulting product then is dried in a freeze drier.

### III. Method of Making and Characterizing pH Sensitive Compositions

As discussed, the invention provides a method of producing pH sensitive compositions for pH targeted drug delivery. In one approach, the method comprises (a) producing a solution comprising pH sensitive diblock copolymers, for example, the copolymers discussed in Section II, and a water insoluble pharmaceutically active agent; and (b) drying the solution of step (a) to produce a dried product. The drying can be facilitated by a number of techniques in the art including, for example, freeze drying, spray drying, and fluid bed drying.

In certain embodiments the solution produced in step (a) has a pH greater than about 7. Accordingly, under certain circumstances the method further includes the step of, after step (a) but before step (b), adjusting the pH of the solution to a pH from about 5 to about 7, for example, to about pH 6. In other embodiments, the pH of the diblock copolymer containing solution is adjusted to a pH from about pH 5 to about pH 7 before the water insoluble pharmaceutically active agent is added.

In certain embodiments, it is understood, that prior to step (a), the pH sensitive diblock copolymers and the water insoluble pharmaceutically active agent are separately dissolved in two separate and distinct portions of the same solvent. After solubilization, the solutions are combined to produce the solution of step (a). In certain other embodiments, it is understood, that prior to step (a), the pH sensitive diblock copolymers and the water insoluble pharmaceutically active agent are dissolved in two separate solvents for example, an organic solvent and an aqueous solvent, before they are mixed together. After solubilization, the solutions are combined to produce the solution of step (a).

For example, exemplary, aqueous solvents include, for example, water, buffer, alkaline solutions, and salt solutions, for example solutions containing NaCl. In addition, exemplary organic solvents include, for example, dimethylsulfoxide (DMSO), alcohol (for example, methanol, ethanol, propanol, isopropanol, butanol, t-butanol), chloroform, dioxane, tetrahydrofuran, acetone, ethyl acetate, and Class II and Class III solvents.

The resulting micelles typically have an average diameter, as measured by dynamic light scattering, of less than about 1000 nm. Typically the micelles have a size in the range of from about 20 nm to about 950 nm, from about 30 nm to about 750 nm, from about 40 nm to about 600 nm, from about 50 nm to about 500 nm, from about 50 nm to about 950 nm, from about 50 nm to about 750 nm, from about 50 nm to about 600 nm, from about 50 nm to about 400 nm, or from about 50 nm to about 200 nm.

In addition, the pH1 sensitive micelles have a loading capacity ranging, as measured by dynamic light scattering in order to determine particle size distribution contain from about 5% to about 80% by weight of pharmaceutically active agent. In certain embodiments, compositions disclosed herein include more than about 5% by weight of pharmaceutically active ingredient, for example between about 5% and about 80%, or between about 10% and about 60%, or between about 15% and about 40% by weight. Different loading capacities can be achieved by varying the relative amounts of the pharmaceutically active agent and the polymer used during the loading process.

The kinetics of drug release can be determined by measuring the amount of drug released into phosphate buffer pH 6.8 at 37° C. via conventional high pressure liquid chromatography (HPLC).

### IV. Dosing and Administration

Under certain circumstances, the dried composition produced in Section III can be administered directly to a mammal, for example, a human, as a solid dosage form, for example, in the form of a powder, cake or a tablet. Alternatively, prior to use, the dried product can be reconstituted into a physiologically acceptable solution, for example, water, a saline solution or a dextrose solution, to produce a solution or suspension.

It is understood that the dose and mode of administration can vary to a large extent depending upon the required needs of the patient, the pharmacokinetics of the active ingredient, and the specific requirements of the treating physician. For example, the dosage of any compositions of the present invention will vary depending on the symptoms, age and body weight of the patient, the nature and severity of the disorder to be treated or prevented, the route of administration, and the form of the subject composition.

The compositions of the invention are designed to provide a therapeutically effective amount of the pharmaceutically active agent. The phrase “therapeutically effective amount” means an amount of a substance that produces some desired local or systemic effect at a reasonable benefit/risk ratio applicable to any treatment. For example, certain compositions of the present invention may be administered in a sufficient amount to produce an amount at a reasonable benefit/risk ratio applicable to such treatment.

Generally, a therapeutically effective amount of dosage of active component will be in the range of from about 0.1 to about 100 mg/kg of body weight/day, or from about 0.5 to about 75 mg/kg of body weight/day, or from about 1.0 to about 50 mg/kg of body weight/day. Dosages for the compositions of the present invention may be readily determined by techniques known to those of skill in the art. The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like.

Also, it is understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

It is understood that the compositions of the invention can be administered orally or parenterally. Parenteral modes of administration include, for example, topically, transdermally, subcutaneously, intravenously, intramuscularly, intrathecally, rectally, vaginally and intranasally. In addition to being administered as single or as multiple doses, it is contemplated that, depending on the mode of administration, the compositions can be administered as a bolus or as an infusion.

It is understood that compositions described herein, although useful for treating a medical disorder, are particularly effective in the treatment of cancer, for example, a tumor, neoplasm, lymphoma or leukemia. It is understood that the compositions of the invention can be used to treat or ameliorate the symptoms of cancer of the colon, lung, prostate, breast, brain, skin, head and neck, liver, pancreas, bone, testicles, ovaries, cervix, kidney, stomach, esophagus, and leukemias and sarcomas. It is contemplated that the SN-38 containing micelles will be particularly effective in treating colorectal cancer, for example, metastatic colorectal cancer. Furthermore, it has been discovered that SN-38, when delivered via the micellar formulations of the invention, results in the formation of lower amounts of at least one metabolite, in particular a glucoronidate metabolite which is known to be toxic, than when SN-38 is administered as a prodrug, commercially available under the trade name Camptosar® (See Example 6).

The invention will now be illustrated by means of the following examples which are given for the purpose of illustration only and without any intention to limit the scope of the present invention.

### EXAMPLES

### Example 1

### Synthesis of Exemplary Diblock Copolymers

This Example describes a protocol for making polyethyleneglycol-b-[poly(n-butylmethacrylate)-co-poly-(acrylic acid)] (PEG-PMA).

Briefly, polyethylene glycol methyl ether (PEG-ME) (Aldrich Chemicals, Oakville, Ontario) was dried by azeotropic distillation with toluene just before use. Potassium hydride (KH) (Aldrich Chemicals, Oakville, Ontario) 30 wt. % in mineral oil, tert-Butyl methacrylate (t-BMA) and n-butyl methacylate (n-BMA) were purified by cryo-distillation before use.

SCHEME 1 shows the synthesis of the intermediate PEG-block-P(nBMA-co-tBMA).

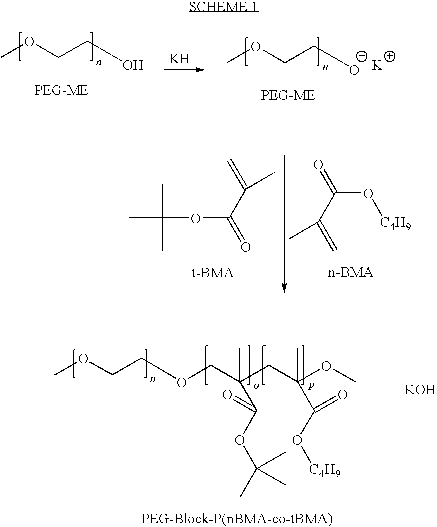

20.0 g PEG (MW 2,000, (10.0 mmol) was dried by azeotropic distribution with 150 ml, toluene (bath set at 135° C.). The polymer was further dried at 100° C. under vacuum for 4 hours. After the polymer cooled to room temperature, 600 mg KH (15.00 mmol, 2000 mg (2.0 mL), 30% KH dispersion in mineral oil) was added under Argon atmosphere, and placed under vacuum for 15 minutes. 700 ml, of freshly distilled THF was added to dissolve the polymer. The reaction between KH and PEG was carried out for 2 hours with stirring. Then, freshly distilled 64 mL of t-BMA (56 g MW 142.2, 393.8 mmol) and 36 mL of n-BMA (28.6 g MW142.2, 151.2 mmol) were added to the reaction mixture and the solution was stirred for a further 2 hours at 20° C. for the block copolymerization. The resulting intermediate PEG-block-P(nBMA-co-tBMA) polymer was collected following solvent evaporation.

SCHEME 2 shows the conversion of the intermediate PEG-block-P(nBMA-co-tBMA) into the pH sensitive PEG-PMA diblock copolymer.

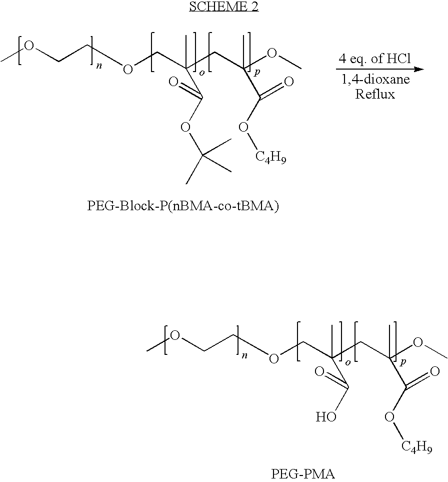

Briefly, 700 ml of 1,4-dioxane and 4 equivalents of HCl (≈1900 mmol HCl≈162 mL HCl_{conc}) were added to the product of SCHEME 1. After addition, the mixture was refluxed overnight. After the solution cooled to room temperature, the solvent was removed. The resulting product was dissolved in THF and concentrated to about 200 mL. The mixture was purified by precipitation in cold water (about 2000 mL) and centrifuged at 10,000 rpm for 10 minutes. A white crude product was obtained. The product was redissolved in THF, precipitated with water, and the precipitate harvested by centrifugation. This cycle was repeated once again. Finally, the resulting white crude product was dried by freeze drying. Various batches of PEG-PMA were produced according to this protocol. The resulting PEG-PMA polymers were characterized.

The composition of 13 different batches of polymer are summarized in TABLE 1.

TABLE 1

| | Degree of Polymerization by NMR¹ | | | NMR² | SLS³ |
|---|---|---|---|---|---|
| Sample | PEG | MAA | n-BMA | M_{w} | M_{w} |
| 1 | 45 | 69 | 25 | 11489 | 11200 |
| 2 | 45 | 69 | 25 | 11489 | 11900 |
| 3 | 45 | 47 | 31 | 10450 | 14100 |
| 4 | 45 | 57 | 27 | 10741 | 10400 |
| 5 | 45 | 69 | 27 | 11773 | 12400 |
| 6 | 45 | 108 | 37 | 16550 | 10700 |
| 7 | 45 | 79 | 28 | 12776 | 10400 |
| 8 | 45 | 101 | 39 | 16232 | 10100 |
| 9 | 45 | 59 | 27 | 10913 | 15300 |
| 10 | 45 | 61 | 24 | 10659 | 15800 |
| 11 | 45 | 58 | 24 | 10401 | 14300 |
| 12 | 45 | 57 | 24 | 10315 | |
| 13 | 45 | 57 | 26 | 10599 | |

¹The structure of the resulting polymers were characterized by NMR. The Degree of Polymerization (DP) of each comonomer of the PEG-PMA was determined by ¹H NMR Spectroscopy (Bruker 300 MHz).
²The molecular weights of the resulting polymers were derived via ¹H NMR Spectroscopy (Bruker 300 MHz).
³The molecular weights of the resulting polymers were also derived via static light scattering (SLS) of the polymer dissolved in methanol using a Zetasizer (Malvern, UK).

### Example 2

### pH Sensitive Compositions Containing SN-38

This Example describes a protocol for making a pH sensitive drug delivery vehicle for delivering the camptothecin derivative, SN-38.

PEG-PMA polymer produced as described in Example 1 was dissolved in 0.1 M sodium hydroxide (NaOH) to produce a final PEG-PMA concentration of 50 mg/mL. Separately, SN-38 was dissolved in 0.1 M NaOH to a final concentration of 4 mg/mL, which, under these conditions, was yellow in color. The two solutions then were mixed together. The resulting solution was also yellow in color.

The resulting solution then was titrated with HCl or 0.1 M citric acid until the yellow color disappeared. Water then was added until the final concentration of SN-38 was 1 mg/mL. The pH, when measured, typically was between about 5.5 and 7. The drug loading level was about 10% by weight but similar formulations can be prepared at drug loading levels ranging from 5% by weight to 80% by weight by varying the ratio of the active ingredient and polymer used in the loading process.

The resulting solution was divided into vials (about 1 ml of solution per vial) and frozen. The frozen solutions then were freeze dried for about 24 hours in a benchtop manifold freeze drier (Flexidry MP from FTS Systems). The freeze drying produced a dried cake, which could be readily reconstituted as a solution or suspension in aqueous solvent such as phosphate buffer pH 6.8. Once reconstituted, the SN-38 solution/suspension remained in solution for 4 to 24 hours at room temperature.

### Example 3

### Release Kinetics of SN-38 Containing Compositions

The SN-38 containing micelles produced by the method described in Example 2 were characterized as described below.

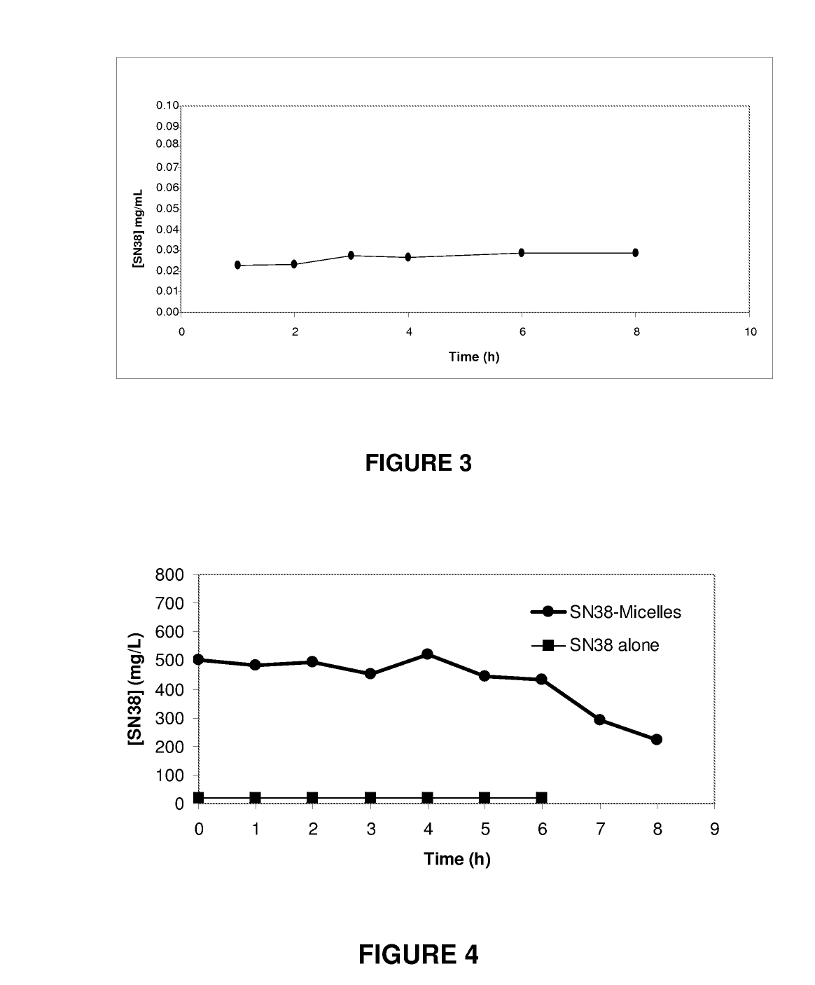

A micellar composition produced in accordance with Example 2 containing 1 mg of SN-38 and 9 mg of PEG-PMA was added to 2 mL of aqueous HCl at pH 1.2. pH 1.2 is about the pH in the human stomach. The rate of drug release was measured via conventional HPLC. The results are presented in FIG. 3, which demonstrate that the SN-38 (——) was not substantially released in the aqueous buffer at pH 1.2, even after eight hours.

The experiment was repeated in a solution at a higher pH, specifically pH 6.8. Briefly, the dissolution of SN-38 was measured either as SN-38 alone or from SN-38 containing micelles prepared as described in Example 2. The freeze dried cake produced in Example 2 was added to phosphate buffer pH 6.8 and the drug concentration was measured under the same conditions as the experiment using aqueous HCl at pH 1.2. The results are summarized in FIG. 4.

FIG. 4 shows that SN-38 dissolves from the micelles (——) within an hour to produce a solution containing 500 mg/L of SN-38 that remains at that concentration for about six hours. In contrast, SN-38 alone (—▪—) rapidly precipitates from solution under the same conditions. The micellar composition of the invention at pH 6.8 was found to have an average particle size of about 50-200 nm as measured by static light scattering using a Zetasizer (Malvern, UK).

### Example 4

### Pharmacokinetic, Toxicity and Efficacy Studies with SN-38 Containing Compositions

This example includes a series of experiments that demonstrate that SN-38 can be delivered in vivo using the micellar compositions of the invention.

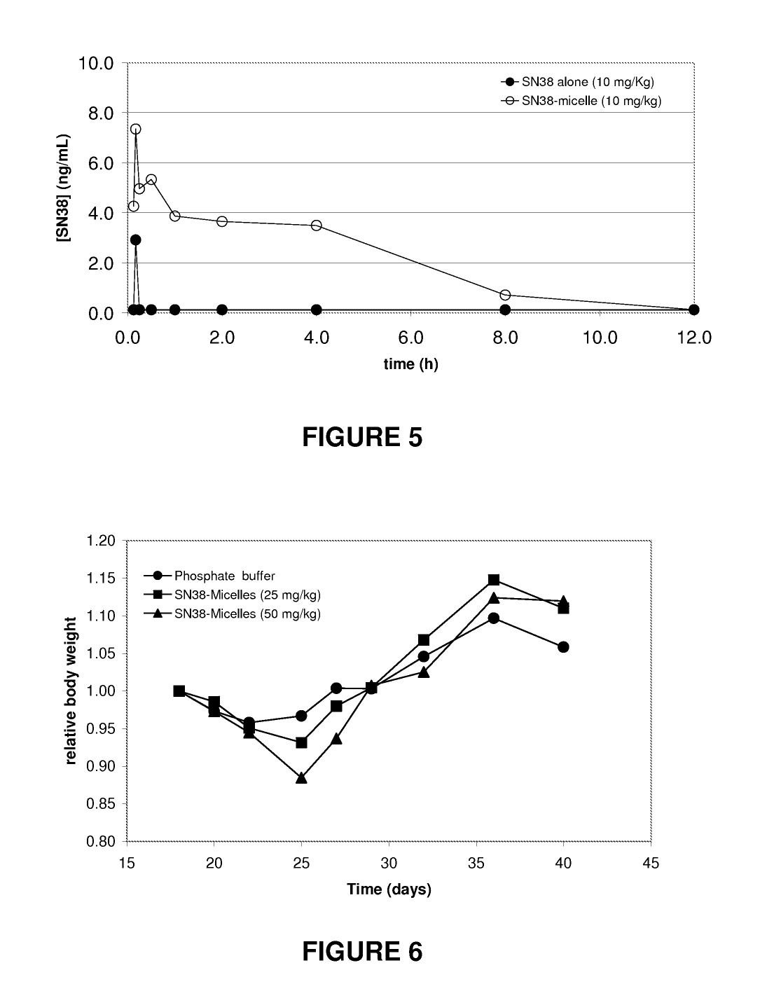

A. In one experiment, 10 mg/kg of SN-38 alone or SN-38 containing micelles were orally administered to two groups of mice (six mice per group). For SN-38 alone, the SN-38 was administered in water. For SN-38 micelles, the SN-38 micelles were administered in phosphate buffer pH 6.8. Plasma samples were harvested at different time points after administration and the drug concentration measured. The results are shown in FIG. 5, where the plasma concentration of SN-38 released from the micellar composition is denoted by —∘— and from SN-38 alone is denoted by ——. The results demonstrate that SN-38 could be delivered from the micellar compositions of the invention. In contrast, the SN-38 provided alone did not appear to be delivered to the plasma.

B. In another experiment, toxicity studies were performed on Swiss nude mice bearing HT-116 tumor cells (human colon cancer cells). Three groups of mice (3 animals per group) were administered orally with either phosphate buffer, 25 mg/kg SN-38 containing micelles, 50 mg/kg SN-38 containing micelles or 100 mg/kg SN-38 containing micelles. The relative body weights of the animals were measured over time. The results are summarized in FIG. 6, which show that doses of 25 mg/kg and 50 mg/kg were well tolerated by the HT-116 tumour bearing mice. The 100 mg/kg dose was less well tolerated as the mice lost 20% of body weight and certain of the mice died.

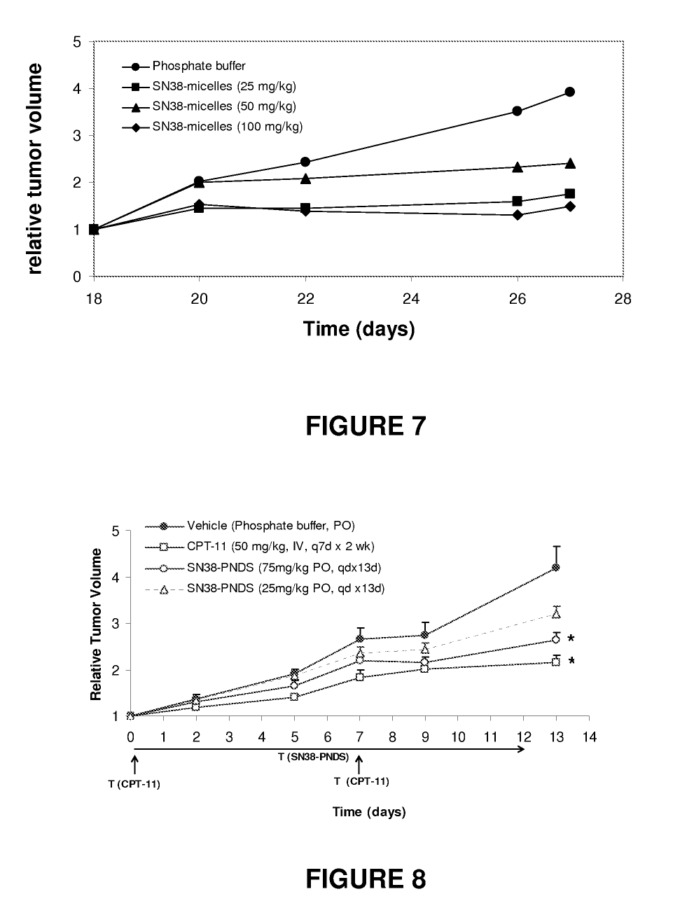

While the body weights of the mice under study were recorded over time, the size of the tumors were also measured in order to provide an indication of the efficacy of the formulation according to the invention. The results are shown in FIG. 7, which show that all the groups (n=3) containing SN38-micelles (—▪—) 25 mg/kg SN-38 micelles; (—▴—) 50 mg/kg SN-38 micelles; —♦— 100 mg/kg SN-38 micelles) showed slower tumor progression than the control group receiving only a phosphate buffer (——).

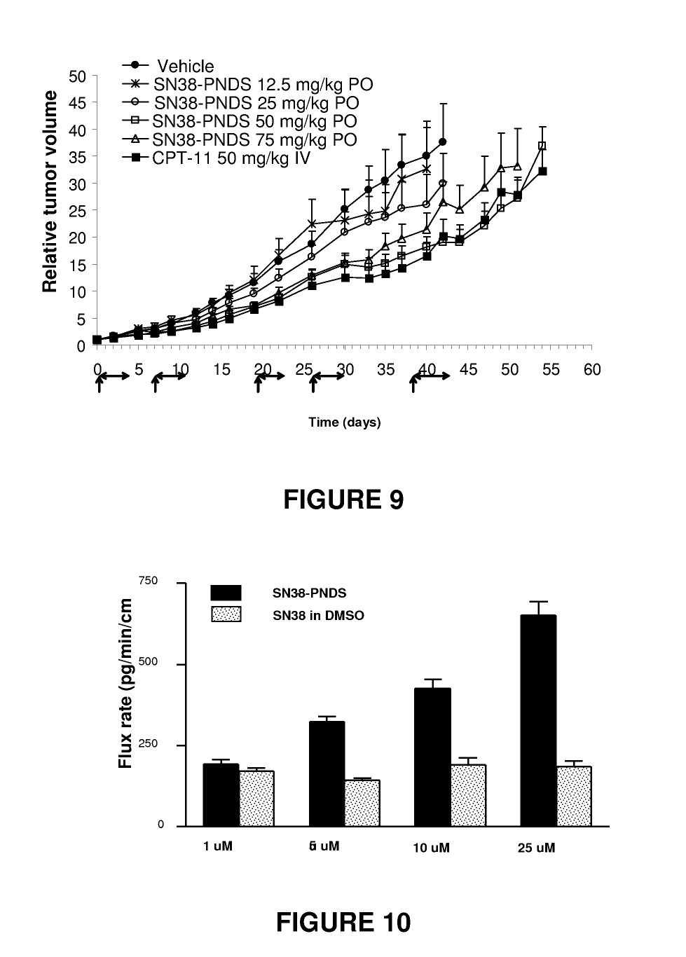

C. In a third experiment, SN-38 micelles prepared according to Example 2 were administered orally as repeated doses to Swiss nude mice bearing HCT-116 human colon xenografts. In a first dosing regimen (regimen 1), mice (n=21) were dosed by oral gavage (PO) for 13 days receiving daily doses of either 25 or 75 mg of SN-38/kg/administration. In a second dosing regimen (regimen 2), mice (n=12) were dosed with 12.5, 25, 50, or 75 mg/kg SN-38 micelles five times per week for two weeks every 21 days (25 administrations total). Intravenous irinotecan (CPT-11), which is among the most widely used chemotherapy agents for treating metastatic colorectal cancer, was used as a positive control at 50 mg/kg/week (n=10 for regimen 1, n=12 for regimen 2). In regimen 1, CPT-11 was administered by intravenous injection on days 0 and 7. In regimen 2, CPT-11 was administered on days 0, 7, 19, 26, and 38. Body weights, tumor volumes, signs of toxicity, and survival were recorded. The results from regimen 1 are shown in FIG. 8, and the results form regimen 2 are shown in FIG. 9. In both figures, the vertical arrows on the time axis represent CPT-11 treatment and the horizontal arrows represent treatment with SN-38 micelles.

Mice receiving SN-38 micelles at a dosage of 75 mg/kg/administration under regimen 1 experienced significant reductions in relative tumor growth compared with vehicle controls (P<0.05) (FIG. 8). There were no overt signs of toxicity (body weight loss, diarrhea); 10% of animals died or were sacrificed during the study. Under regimen 2, animals receiving SN-38 micelles at a dosage of 50 and 75 mg/kg both demonstrated significant (p<0.05) tumor growth reductions compared to vehicle controls (FIG. 9). The formulation was well tolerated with no overt signs of toxicity and no deaths. In both studies, SN-38 micelles administered orally at 50 and 75 mg/kg doses demonstrated reduced tumor growth equivalent (P>0.05) to weekly intravenous injections of CPT-11.

D. In a fourth experiment, the pharmacokinetics of the SN-38 micelles were measured in rats. Briefly, 50 mg/kg of SN38-PEG-PMA (SN-38 micellar formulation) in phosphate buffer, pH 6.8, was administered orally to female Sprague-Dawley rats (n=8). As a control, 15 mg/kg of SN-38 in PBS buffer was orally administered to a corresponding population of rats. At different time points, plasma samples were harvested and the concentrations of SN-38 were determined by HPLC using fluorescence detection. The results are summarized in TABLE 2.

TABLE 2

| | SN38-PEG-PMA | SN38 |
|---|---|---|
| AUC (ng/mL*h) | 30 ± 14 | 0 |
| Cₘₐₓ (ng/mL) | 43 ± 20 | ND |
| Tₘₐₓ (h) | 0.15 ± 0.06 | ND |
| T_{1/2} (h) | 0.7 ± 0.5 | ND |

ND: not detected

As shown in TABLE 2, SN-38 was detected in the SN38-PEG-PMA group but not in the SN-38 solution group. These results demonstrate the effectiveness of SN-38 dosing using micellar compositions of the invention.

### Example 5

### Permeability of Human Colon Carcinoma Cells

The purpose of this Example was to assess the uptake of SN-38 by human colon cells (Caco-2 colon carcinoma cells) in vitro.

Layers of Caco-2 cells were prepared as follows. The Caco-2 cells were seeded at a density of approximately 60,000 cells/cm² onto collagen-coated, microporous, polycarbonate membranes in 12-well Transwell® plates. The cells were maintained in high glucose (4.5 g/L) DMEM, supplemented with 10% fetal bovine serum (FBS), 1% nonessential amino acids (NEAA), 1% L-glutamine, penicillin (100 U/mL), and streptomycin (100 μg/mL) at 37° C. in a humidified incubator with 5% CO₂. The culture medium was changed 24 hours after seeding to remove cell debris and dead cells. Afterwards the medium was changed every other day for three weeks. Prior to the transport experiment, each batch of cell monolayers was certified by transepithelial electric resistance (TEER) measurement and by permeability determination of the control compounds, propranolol (10 μM), pindolol (10 μM), atenolol (10 μM), and digoxin (5 μM). The permeability assay buffer I (pH 7.4) was Hanks Buffer Salt Solution (HBSSg) containing 15 mM D(+)glucose and 10 mM HEPES, pH 7.4±0.1. The assay buffer II (pH 6.5) was HBSSg containing 15 mM D(+)glucose and 10 mM MES, pH 6.5±0.1. The apparatus was incubated at 37° C. with 5% CO₂ in a humidified incubator during the assay period. The Caco-2 cells were washed twice with the washing buffer (HBSS containing 10 mM HEPES and 15 mM glucose at pH 7.4).

In a first study, the SN-38 micellar compositions prepared in accordance with Example 2 were dissolved in HBSS buffer (either buffer I—pH 7.4 or buffer II—pH 6.5) to create solutions containing either 1.0 mg/L SN-38 or 10 mg/L SN-38. The solutions were applied to a first reservoir (donor reservoir) adjacent the monolayer and HBSS buffer was placed in a second reservoir (recipient reservoir) adjacent the monolayer. The transport of the SN-38 was measured using an Endothelin-12 resistance meter (World Precisions, Boston, Mass.). The results are summarized in TABLE 3.

TABLE 3

| SN-38 | Donor | | |
|---|---|---|---|
| Concentration | Reservoir | Flux rate | Permeability* |
| (mg/L) | pH | (pg/min/cm²) | (×10⁻⁶ cm/sec) |
| 1.0 | 6.5 | 34.59 ± 3.85 | 0.64 ± 0.07 |
| 10 | 6.5 | 56.90 ± 15.41 | 0.11 ± 0.03 |
| 1.0 | 7.4 | 33.33 ± 5.95 | 0.58 ± 0.10 |
| 10 | 7.4 | 63.80 ± 11.04 | 0.12 ± 0.02 |

The results set forth in TABLE 3 show that SN-38 can permeate through the Caco-2 cell layer when formulated in pH sensitive micelles. Based on this data, it is contemplated that the SN-38 will also be absorbed in vivo, consistent with the rodent studies in Example 4.

In a second study, SN-38 micellar compositions prepared in accordance with Example 2 were dissolved in Hank's buffer pH 6.8. Non-formulated SN-38 was dissolved in 0.05% DMSO in Hank's buffer. Four concentrations of each SN-38 solution were prepared: 1 μM, 5 μM, 10 μM and 25 μM. The transport of SN-38 either formulated in micelles (SN-38 PNDS) or non-formulated SN-38 dissolved in DMSO from the apical to the basolateral side of the (Caco-2 monolayer was evaluated after 120 minutes at 37° C. at each concentration. The results are summarized in FIG. 10. As shown in FIG. 10, the flux of SN-38 from the micellar formulation (SN-38 PMDS) across the monolayer increased as a function of SN-38 concentration. In contrast, the permeability of SN-38 in DMSO was approximately the same and did not increase with an increase in concentration. These results demonstrate the effectiveness of SN-38 micelles to deliver SN-38 across a membrane, for example, an intestinal wall membrane.

### Example 6

### Pharmacokinetic Studies of SN-38 and its Metabolites

This Example demonstrates that when SN-38 is formulated into micelles, the production of at least one toxic metabolite SN-38 glucoronidate is decreased relative to when SN-38 is administered as a prodrug in a commercially available formulation known as Camptosar® (CPT-11), which contains the active ingredient irinotecan.

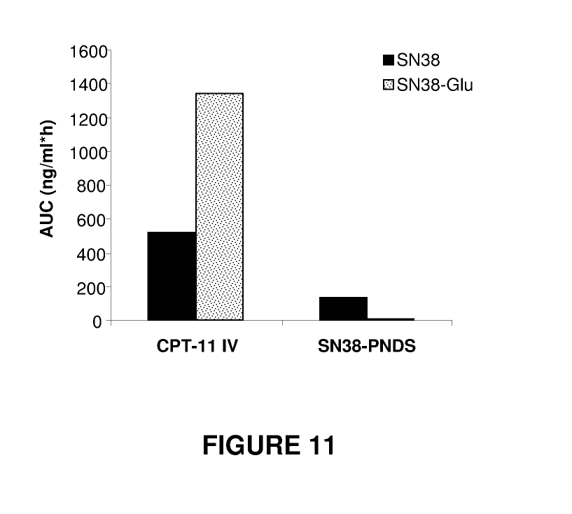

50 mg/kg of SN-38 micellar formulation prepared in accordance with Example 2 was orally administered to female Sprague-Dawley rats (n=8). As a control, 6.2 mg/kg of irinotecan (CPT-11) was administered intravenously to a corresponding population of rats. At different time points, plasma samples were harvested and the concentrations of SN-38 and SN-38 glucoronidate (SN-38 Glu) were determined. The results are summarized in FIG. 11.

As shown in FIG. 11, intravenous administration of CPT-11 resulted in considerable SN-38 metabolism to produce SN-38 Glu (AUC_{SN-38 Glu}/AUC_{SN-38}=2.60). In contrast, when SN-38 micelles were administered orally, negligible conversion of SN-38 to SN-038 Glu was observed (AUC_{SN-38 Glu}/AUC_{SN-38}=0.03). Because SN-38 Glu is a toxic metabolite formed in the liver, it is contemplated that the micellar compositions of the invention may reduce the toxicity associated with SN-38 administration.

### Example 7

### Compositions Containing Docetaxel

This Example describes the preparation of pH sensitive docetaxel containing formulations. Briefly, PEG-PMA, as prepared in Example 1, was dissolved in 0.1 M NaOH to give a final concentration of 50 mg/ml. Separately, docetaxel was dissolved in t-butanol at a concentration of 20 mg/mL. A colorless solution was obtained. The two solutions were mixed together to give a colorless solution.

The resulting mixture was titrated with 0.1 M citric acid until the pH was between about 5.8 and about 6.5. Water was added until the final concentration of docetaxel was 1 mg/mL. The pH was found to be between 5.5 and 7.0, and the drug loading level ranged from 5% to 20%.

The solution was divided into vials containing 1 to 18 mL of solution, which were then frozen at −60° C. in a freeze dryer. The frozen solution was freeze dried over three days. A dry cake was obtained which could be reconstituted in phosphate buffer, pH 6.8. It was found that docetaxel remained in solution for more than 6 hours at 37° C.

### Example 8

### Compositions Containing Paclitaxel

This Example describes the preparation of pH sensitive paclitaxel containing formulations. Briefly, PEG-PMA, prepared as described in Example 1, was dissolved in 0.1 M NaOH to give a final concentration of 50 mg/ml. Separately, paclitaxel was dissolved in t-butanol to give a final concentration of 8 mg/mL. The two solutions were mixed together to produce a colorless solution. The resulting solution was titrated with 0.1 M citric acid until the pH was between 5.8 and 6.5. Water was added until the final concentration of paclitaxel was 1 mg/mL, and the pH of the solution was found to be between about 5.5 and about 7. The drug content varied between 5 and 40% by weight.

The solution was divided into vials containing 1 to 18 mL of solution which were frozen at −60° C. in a freeze dryer. The frozen solution was freeze dried for 3 days. A dry cake was obtained which could readily be reconstituted in water. Under the conditions tested, paclitaxel remained in solution for more than 6 hours at room temperature.

### INCORPORATION BY REFERENCE

The entire disclosure of each of the patent and scientific documents referred to herein is incorporated by reference for all purposes.

### EQUIVALENTS

Although the present invention has been illustrated by means of preferred embodiments thereof, it is understood that the invention intends to cover broad aspects thereof without departing from the spirit and scope of the invention as defined in the appended claims.

1. A composition for the pH targeted delivery of a water insoluble pharmaceutically active agent, the composition comprising:
(a) a plurality of pH sensitive diblock copolymers; and
(b) a water insoluble pharmaceutically active agent associated with the diblock copolymers, such that, when the composition contacts an aqueous solution at a pH of about 2, less than about 10% of the pharmaceutically active agent is released from the composition after 2 hours, but in an aqueous solution at a pH of about 6 or higher, at least 60% of the pharmaceutically active agent is released from the composition within 2 hours.

2. The composition of claim 1, wherein a first block of the diblock co-polymer comprises monomers selected from the group consisting of poly(ethylene glycol) and poly(vinylpyrrolidone).

3. The composition of claim 1, wherein a second block of the diblock co-polymer comprises (i) ionizable monomeric subunits selected from the group consisting of methacrylic acid, and acrylic acid, and (ii) hydrophobic monomers selected from the group consisting of methacrylate and derivatives thereof, acrylates and derivatives thereof, methacrylamides and acrylamides.

4. The composition of claim 1, wherein the diblock copolymers are defined by Formula I, 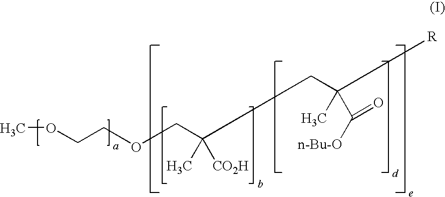
wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0.

5. A pH-sensitive micellar composition for the pH targeted delivery of a water insoluble pharmaceutically active agent, the composition comprising:
(a) micelles comprising a plurality of pH sensitive diblock copolymers; and
(b) a water insoluble pharmaceutically active agent disposed within the micelles,
wherein, in an aqueous solution at a pH of about 2, less than about 10% of the pharmaceutically active agent is released from the micellar composition after 2 hours, but at a pH of about 6 or higher, at least 60% of the pharmaceutically active agent is released from the micellar composition within 2 hours.

6. The composition of claim 5, wherein the diblock copolymers are defined by Formula I, 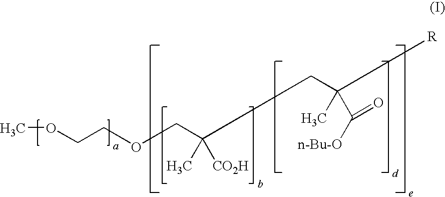
wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0.

7. The composition of claim 1, wherein at a pH of about 6 or higher, at least 70% of the pharmaceutically active agent is released from the composition within 2 hours.

8. The composition of claim 1, wherein at a pH of about 6 or higher, at least 80% of the pharmaceutically active agent is released from the composition within 2 hours.

9. The composition of claim 1, wherein the pharmaceutically active agent is an-anti cancer agent.

10. The composition of claim 9, wherein the anti-cancer agent is a camptothecin derivative.

11. The composition of claim 10, wherein the camptothecin derivative is SN-38.

12. The composition of claim 5, wherein the micelles have an average diameter in the range of from about 20 nm to about 950 nm.

13. The composition of claim 12, wherein the micelles have an average diameter in the range of from about 50 nm to about 200 nm.

14. A composition comprising:
(a) a plurality of pH sensitive diblock copolymers, wherein the diblock copolymers are defined by Formula I, 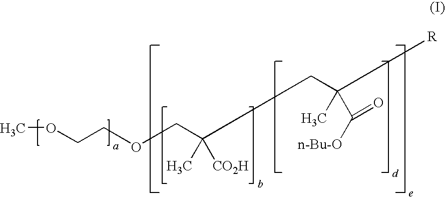
wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0; and
(b) a camptothecin derivative associated with the diblock copolymers.

15. The composition of claim 14, wherein the camptothecin derivative is SN-38.

16. A method of producing a composition of claim 1 for the pH targeted delivery of a water insoluble pharmaceutically active agent, the method comprising:
(a) producing a solution comprising pH sensitive diblock copolymers and the pharmaceutically active agent; and
(b) drying the solution of step (a) to produce a dried product.

17. The method of claim 16, wherein the solution produced in step (a) has a pH greater than about 7.

18. The method of claim 16, further comprising the step of, after step (a) but before step (b), adjusting the pH of the solution to a pH from about 5 to about 7.

19. The method of claim 18, wherein the pH is reduced to about 6.

20. The method of claim 16, wherein the solution produced in step (a) has a pH of from about 5 to about 7.

21. The method of claim 16, wherein, prior to step (a), the pH sensitive diblock copolymers and the pharmaceutically active agent are separately dissolved in two separate portions of the same solvent.

22. The method of claim 16, wherein, prior to step (a), the pH sensitive diblock copolymers and the pharmaceutically active agent are dissolved in different solvents.

23. The method of claim 16, wherein, in step (a), the pharmaceutically active agent is an anti-cancer agent.

24. The method of claim 23, wherein the anti-cancer agent is a camptothecin derivative.

25. The method of claim 24, wherein the camptothecin derivative is SN-38.

26. The method of claim 16, wherein the pH sensitive diblock copolymers subunits are defined by Formula I, 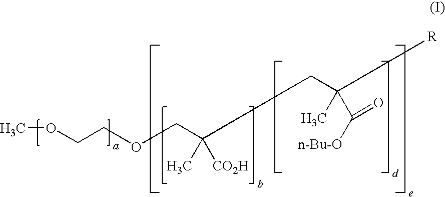
wherein,
R is H, alkyl, hydroxyl, alkoxyl, or halogen,
a is an integer in the range of about 20 to about 60,
b represents independently for each occurrence an integer in the range of 0 to about 20,
d represents independently for each occurrence an integer in the range of 0 to about 20,
e is an integer in the range of about 10 to about 50, and
provided that at least one occurrence of b is >0, and at least one occurrence of d is >0.

27. The method of claim 16, further comprising the step of reconstituting the dried product in a physiologically acceptable solution.

28. A composition produced by the method of claim 16.

29. A method of administering a water insoluble pharmaceutically active agent to a mammal in need of the pharmaceutically active agent, the method comprising administering to the mammal an effective amount of the pharmaceutically active agent in the composition of claim 1.

30. The method of claim 29, wherein the composition is administered orally.

31. The method of claim 29, wherein the composition is administered parenterally.

32. A method of treating cancer in a mammal, the method comprising administering an effective amount of an anti-cancer agent to the mammal in the composition of claim 9.
